(19) United States
(12) Patent Application Publication
(10) Pub. No.: US 2022/0395275 A1
(43) Pub. Date: Dec. 15, 2022

(54) SURGICAL FASTENING INSTRUMENT

(71) Applicant: Covidien LP, Mansfield, MA (US)

(72) Inventors: Olesea Diaz-Chiosa, Naugatuck, CT (US); Osvaldo A. Barrera, Madison, CT (US)

(21) Appl. No.: 17/345,791

(22) Filed: Jun. 11, 2021

(51) Int. Cl. A61B 17/115

(52) U.S. Cl. CPC A61B 17/1155; A61B 2017/1132

(57) ABSTRACT

A surgical fastening device includes a handle assembly, an elongated portion, a shell assembly, a screw drive, and a firing mechanism. The elongated portion extends distally from the handle assembly. The shell assembly is disposed adjacent a distal portion of the elongated portion, and includes a cartridge assembly and an end effector. The screw drive defines a longitudinal axis. A distal portion of the screw drive is engaged with the end effector. Rotation of the screw drive about the longitudinal axis relative to the elongated portion causes longitudinal translation of the end effector relative to the cartridge assembly. The firing mechanism is disposed at least partially within the end effector. A predetermined amount of proximal movement of the end effector relative to the cartridge assembly is configured to at least partially form fasteners disposed in the cartridge assembly.

### BACKGROUND

### Technical Field

The present disclosure relates generally to surgical instruments for applying surgical fasteners to body tissue, and to methods of joining hollow tissue. More particularly, the present disclosure relates to a surgical fastening instrument suitable for performing anastomosis and/or treatment to internal walls of hollow tissue organs, and to methods of performing a transanal anastomosis procedure.

### Background of Related Art

Anastomosis is the surgical joining of separate hollow organ sections. Typically, an anastomosis procedure follows surgery in which a diseased or defective section of hollow tissue is removed, and the end sections are stapled via a linear surgical stapler. Depending on the desired anastomosis procedure, the end sections may be joined by either circular, end-to-end, or side-to-side organ reconstruction methods.

In a circular anastomosis procedure, the two ends of the organ sections are joined by means of a stapling instrument which drives a circular array of staples through the end section of each organ section and simultaneously cores any tissue interior of the driven circular array of staples to free the tubular passage. Examples of instruments for performing circular anastomosis of hollow organs are described in U.S. Pat. Nos. 6,053,390; 5,588,579; 5,119,983; 5,005,749; 4,646,745; 4,576,167; and 4,473,077, each of which is incorporated herein in its entirety by reference. Typically, these instruments include an elongated shaft having a handle portion at a proximal end to actuate the instrument and a staple holding component disposed at a distal end. After being positioned within tissue, an anvil assembly including an anvil rod with attached anvil head is mounted to the distal end of the instrument adjacent the staple holding component.

Opposed end portions of tissue of the hollow organ(s) to be stapled are clamped between the anvil head and the staple holding component. The clamped tissue is stapled by driving one or more staples from the staple holding component so that the ends of the staples pass through the tissue and are deformed by the anvil head. An annular knife is advanced to core tissue within the hollow organ to free a tubular passage within the organ. Generally, both the actuation of the staple forming mechanism and the advancement of the knife occur at the same time.

Besides anastomosis of hollow organs, surgical stapling devices for performing circular anastomosis have been used to treat internal hemorrhoids in the rectum. Typically, during use of a circular stapling device for hemorrhoid treatment, the anvil head and the staple holding component of the surgical stapling device are inserted through the anus and into the rectum with the anvil head and the staple holding component in an open or unapproximated position. Thereafter, a pursestring suture is used to pull the internal hemorrhoidal tissue towards the anvil rod. Next, the anvil head and the staple holding component are approximated to clamp the hemorrhoid tissue between the anvil head and the staple holding component. The circular stapling device is fired to remove the hemorrhoidal tissue and staple the cut tissue.

### SUMMARY

The present disclosure relates to a surgical fastening device including a handle assembly, an elongated portion, a shell assembly, a screw drive, and a firing mechanism. The elongated portion extends distally from the handle assembly. The shell assembly is disposed adjacent a distal portion of the elongated portion, and includes a cartridge assembly and an end effector. The screw drive defines a longitudinal axis. A distal portion of the screw drive is engaged with the end effector. Rotation of the screw drive about the longitudinal axis relative to the elongated portion causes longitudinal translation of the end effector relative to the cartridge assembly. The firing mechanism is disposed at least partially within the end effector. A predetermined amount of proximal movement of the end effector relative to the cartridge assembly is configured to at least partially form fasteners disposed in the cartridge assembly.

In aspects, the firing mechanism may include a ball ring having a plurality of balls, and a wedge ring having a plurality of wedges. In aspects, a tip of each wedge of the plurality of wedges of the wedge ring may be positionable between adjacent balls of the plurality of balls of the ball ring. In disclosed aspects, the wedge ring may be rotatable about the longitudinal axis relative to the ball ring. In aspects, rotation of the wedge ring relative to the ball ring may cause a tip of at least one wedge of the plurality of wedges to move from a first position between adjacent balls of the plurality of balls, to a second position in contact with a distal end of one ball of the plurality of balls.

In aspects, each ball of the plurality of balls may be movable from a first position where each ball of the plurality of balls is free from contact with adjacent balls of the plurality of balls, to a second position where each ball of the plurality of balls contacts two adjacent balls of the plurality of balls. In disclosed aspects, each ball of the plurality of balls may be biased toward the second position. In aspects, each wedge of the plurality of wedges may be fixed from movement relative to adjacent wedges of the plurality of wedges.

In aspects, the surgical fastening device may include a tube extending at least partially through the elongated portion. An external surface of the end effector may include a plurality of distal vacuum slots disposed in fluid engagement with the tube, and an external surface of the cartridge assembly may include a plurality of proximal vacuum slots disposed in fluid engagement with the tube.

In aspects, the cartridge assembly may include a plurality of fasteners therein. Each fastener of the plurality of fasteners may be configured to be secured to one adjacent fastener of the plurality of fasteners. In aspects, each fastener of the plurality of fasteners may include a base having a curved leg extending therefrom. The curved leg may include a tip and a spike. The tip and the spike of the curved leg may be configured to enter a hole of an adjacent fastener of the plurality of fasteners during formation of the plurality of fasteners.

The present disclosure also relates to a shell assembly of a surgical fastening device. The shell assembly includes a cartridge assembly, an end effector, and a firing mechanism. The cartridge assembly is configured to house a plurality of fasteners, and includes a plurality of proximal vacuum slots. The end effector is configured to move toward and away from the cartridge assembly, and includes a plurality of distal vacuum slots. The proximal vacuum slots are in fluid communication with the distal vacuum slots. The firing mechanism is disposed at least partially within the cartridge assembly, and includes a ball ring having a plurality of balls, and a wedge ring having a plurality of wedges.

In aspects, the wedge ring may be rotatable relative to the ball ring between a first position where a tip of at least one wedge of a plurality of wedges of the wedge ring is between adjacent balls of a plurality of balls of the ball ring, and a second position where the tip of the at least one wedge is in contact with a distal end of one ball of the plurality of balls.

In aspects, the ball ring may include a plurality of balls. Each ball of the plurality of balls may be movable from a first position where each ball of the plurality of balls is free from contact with adjacent balls of the plurality of balls, to a second position where each ball of the plurality of balls contacts two adjacent balls of the plurality of balls. In disclosed aspects, the wedge ring may include a plurality of wedges. Each wedge of the plurality of wedges may be fixed from movement relative to adjacent wedges of the plurality of wedges.

In aspects, the cartridge assembly may include a plurality of fasteners therein. Each fastener of the plurality of fasteners may be configured to be secured to one adjacent fastener of the plurality of fasteners.

The present disclosure also relates to a method of performing an end-to-end anastomosis through a single opening. The method includes inserting a distal portion of a surgical fastening device through an opening to access target tissue, positioning an end effector of the distal portion of the surgical fastening device at least partially within a distal tissue lumen, positioning a cartridge assembly of the distal portion of the surgical fastening device at least partially within a proximal tissue lumen, applying suction through the end effector such that portions of the distal tissue lumen are pulled into engagement with the end effector, applying suction through the cartridge assembly such that portions of the proximal tissue lumen are pulled into engagement with the cartridge assembly, moving the end effector towards the cartridge assembly, compressing target tissue between the cartridge assembly and the end effector, and moving a leg of a fastener disposed within the cartridge assembly through the target tissue and into engagement with an adjacent fastener within the cartridge assembly.

In aspects, the method may include rotating a ball ring within the end effector relative to a wedge ring within the end effector. In disclosed aspects, the ball ring may include a plurality of balls, and the method may further include biasing each ball of the plurality of balls radially inward.

In aspects, the end effector may define an outer dimension, and the method may further include reducing the outer dimension of the end effector, followed by removing the end effector from the single opening.

### DESCRIPTION OF THE DRAWINGS

Various aspects of the present disclosure are illustrated herein with reference to the accompanying drawings, wherein:

FIG. 1 is a perspective view of a surgical fastening instrument according to aspects of the present disclosure;

FIG. 2 is a perspective view of a distal portion of the surgical fastening instrument of FIG. 1 in an open position within tissue;

FIG. 3 is a perspective view of the distal portion of the surgical fastening instrument of IG. 1 in an approximated position within tissue;

FIG. 4 is a side view of fasteners for use with the surgical fastening instrument of FIG. 1 in an unformed orientation;

FIG. 5 is a side view of the fasteners of FIG. 4 in a formed orientation through two layers of tissue;

FIG. 6 is a perspective view of the distal portion of the surgical fastening instrument of FIG. 1 in an initial, approximated position, and illustrating a plurality of fasteners and a firing assembly therein;

FIG. 7 is a perspective view of the distal portion of the surgical fastening instrument of FIG. 1 in an open position, and illustrating the plurality of fasteners and the firing assembly therein;

FIG. 8 is a cross-sectional view of a tissue lumen showing the distal portion of the surgical fastening instrument of FIG. 1 in an approximated position within the tissue lumen, and illustrating the plurality of fasteners formed through tissue;

FIG. 9 is a cross-sectional view of the tissue lumen showing the distal portion of the surgical fastening instrument of IG. 1 in an open position within the tissue lumen after the plurality of fasteners have formed through tissue, and when the firing assembly is in a post-fire position;

FIG. 10 is a cross-sectional view of the tissue lumen showing the distal portion of the surgical fastening instrument of FIG. 1 during withdrawal from the tissue lumen; and

FIGS. 11-13 are schematic views of a portion of the firing assembly of the surgical fastening instrument of FIG. 1 shown in various positions.

### DETAILED DESCRIPTION

Aspects of the presently disclosed surgical fastening instrument will now be described in detail with reference to the drawings wherein like numerals designate identical or corresponding elements in each of the several views. As is common in the art, the term “proximal” refers to that part or component closer to the user or operator, e.g., surgeon or physician, while the term “distal” refers to that part or component farther away from the user.

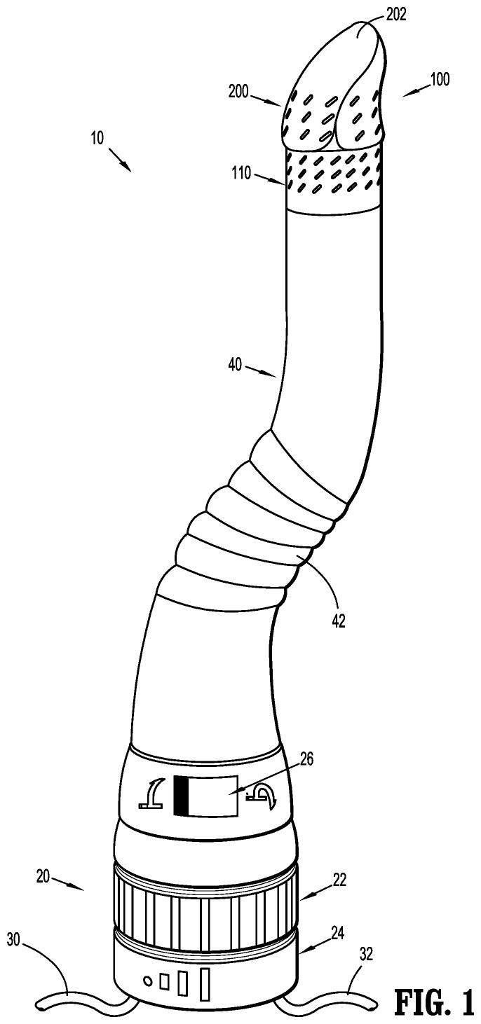

FIG. 1 illustrates an aspect of a surgical fastening instrument according to the present disclosure, referenced generally as numeral 10. Surgical fastening instrument 10 includes a handle assembly 20, an elongated portion 40 extending distally from handle assembly 20, and a shell assembly 100 mounted adjacent a distal end of elongated portion 40. The shell assembly 100 includes a cartridge assembly 110 and an end effector 200.

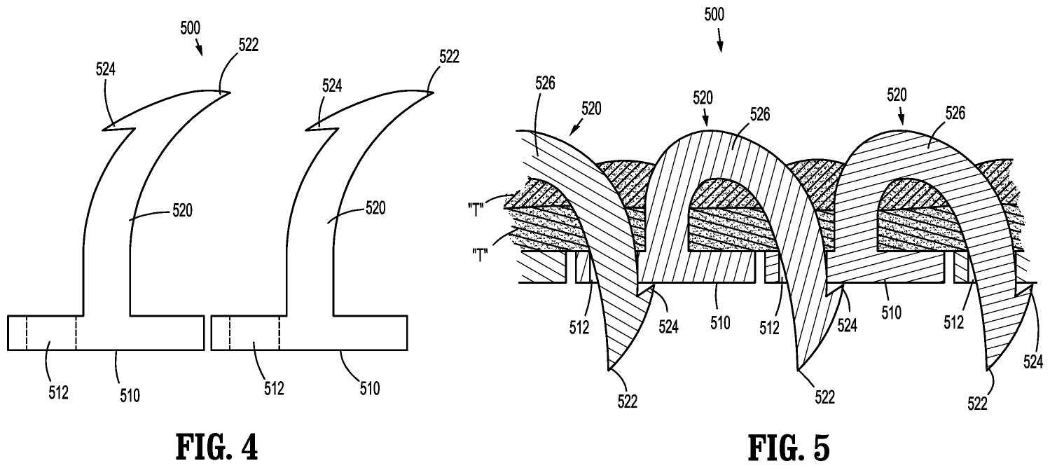

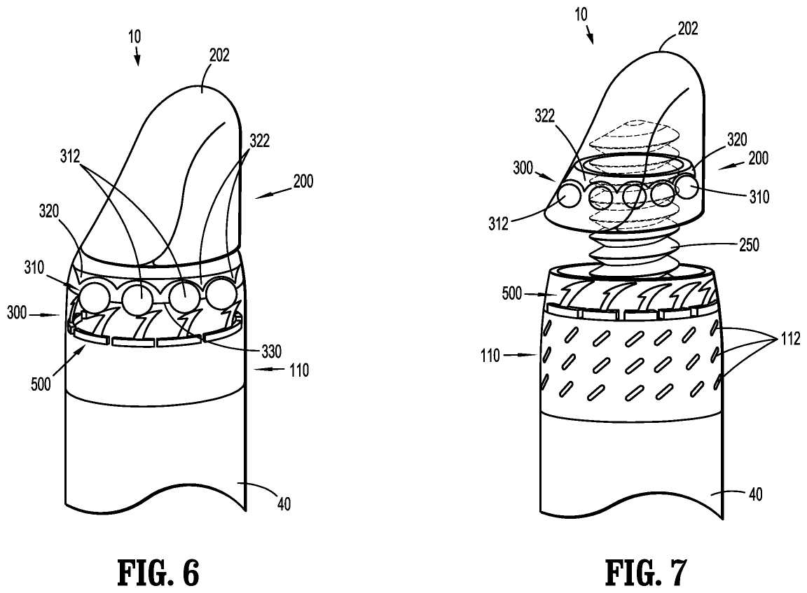

The handle assembly 20 shown in FIG. 1 is a power-operated handle including a first actuator 22, a second actuator 24, and a third actuator 26. While the first actuator 22, the second actuator 24, and the third actuator 26 can be configured to perform at least one function, it is disclosed that the first actuator 22 causes the end effector 200 to move proximally and distally relative to the cartridge assembly 110, the second actuator 24 controls the amount of suction applied to the cartridge assembly 110 and the end effector 200, and the third actuator 26 causes movement of portions of a firing mechanism 300 (FIGS. 6-9) within the end effector 200 to cause formation of fasteners 500 (FIGS. 4-5), for instance. In the illustrated aspect, the handle assembly 20 includes an electrical cord 30 configured to supply the surgical fastening instrument 10 with power, and a hose 32 configured to remove a gas or fluid (e.g., air) from areas surrounding the surgical fastening instrument 10 and through the surgical fastening instrument 10.

It is contemplated that the shell assembly 100 according to the present disclosure can be part of a surgical system. The elongated portion 30 of the surgical fastening instrument 10 may itself be a removable and replaceable part of the system. The handle assembly 20 can be manual, powered, and/or part of a robotic surgical system.

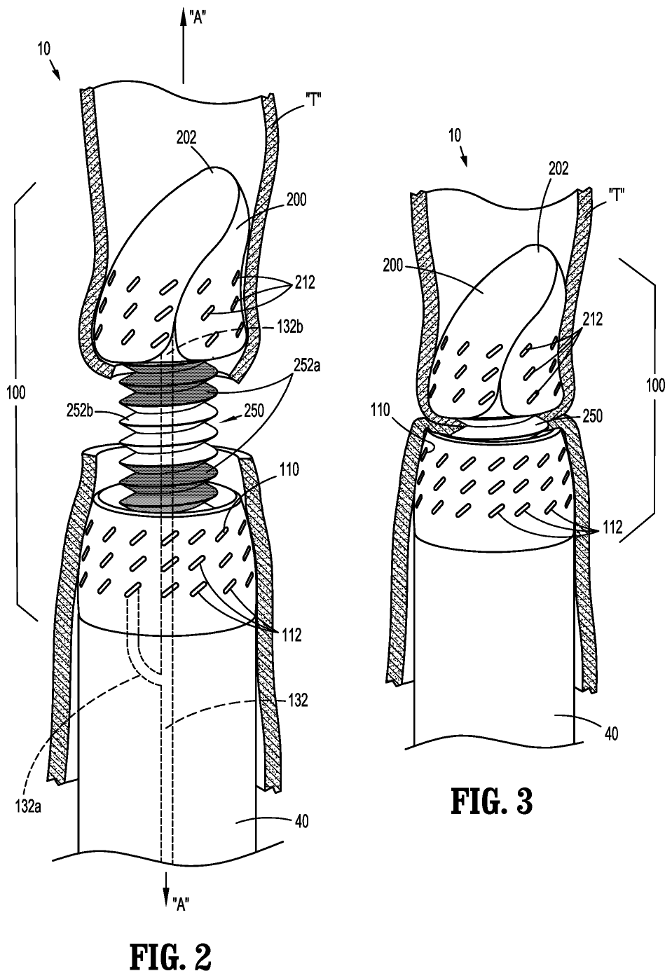

With reference to FIGS. 2 and 3, the shell assembly 100 is configured to be positioned within hollow body tissue “T.” More particularly, in disclosed aspects, the shell assembly 100 and at least part of the elongated portion 40 are configured to be inserted trans-anally and used to repair and/or join tissue during or following a lower anterior resection procedure. The surgical fastening instrument 10, for instance, enables a single user to complete an end-to-end anastomosis through a single bodily opening (i.e., a body orifice or an incision).

The elongated portion 40 includes an articulating pivot 42 (FIG. 1) that is configured to passively articulate during insertion through the tissue lumen. Thus, the articulating pivot 42 helps enable the elongated portion 40 to conform to the anatomical shape of the gastro-intestinal tract, for example, while minimizing pressure applied to the tissue. Moreover, the end effector 200 of the shell assembly 100 includes a tapered distal end 202 that is configured for smooth navigation within the tissue lumen.

With continued reference to FIGS. 2 and 3, the shell assembly 100 includes a screw drive 250 interconnecting the cartridge assembly 110 and the end effector 200. Rotation of the screw drive 250 relative to cartridge assembly 110 causes proximal or distal movement of the end effector 200 relative to the cartridge assembly 110 (depending on the direction of rotation), between a spaced position (FIG. 2) and an approximated position (FIG. 3). Additionally, as shown in FIG. 2, the screw drive 250 includes indicia 252a, 252b (e.g., bands of different colors or patterns) thereon to indicate various degrees of extension (and can be read by a laparoscopic camera, for instance). For example, the visibility (or perceivability) of indicia 252a may indicate that the screw drive 250 has been sufficiently extended for adequate tissue grip. Additionally, the screw drive 250 defines an axis “A-A.”

As is also shown in FIGS. 2 and 3, the cartridge assembly 110 includes a plurality of proximal vacuum slots 112, and the end effector 200 includes a plurality of distal vacuum slots 212. The proximal vacuum slots 112 and the distal vacuum slots 212 are fluidly engaged or in fluid communication with the hose 32 (FIG. 1) through a tube 132 interconnecting the proximal vacuum slots 112 and the distal vacuum slots 212 with the hose 32. More particularly, the proximal vacuum slots 112 are fluidly engaged or in fluid communication with the tube 132 via a first tube leg 132a, and the distal vacuum slots 212 are fluidly engaged or in fluid communication with the tube 132 via a second tube leg 132b, which extends through the screw drive 250. The tube 132, or portions thereof, are flexible enough and have adequate slack to perform sufficiently when the end effector 200 is both in the spaced position and the approximated position, and positions therebetween.

In aspects, an inlet port of the tube 132 is in fluid communication with a ring-like space between the screw drive 250 and an inner wall of the cartridge assembly 110. Additionally, an inlet port of the second tube leg 132b is in fluid communication with a ring-like space between the screw drive 250 and an inner wall of the end effector 200.

With reference to FIGS. 4 and 5, a plurality of fasteners 500 is shown. More specifically, FIG. 4 illustrates unformed fasteners 500, and FIG. 5 illustrates fasteners 500 formed through two layers of tissue “T” and secured to adjacent fasteners 500. With particular reference to FIG. 4, each fastener 500 includes a base 510 having a hole 512, and a leg 520 having a tip 522 and a spike 524. As shown, in the unformed orientation, the leg 520 defines a curved shape, which enables or facilitates the formation of the fastener 500. Moreover, the leg 520 curves away from the hole 512 in the base 510. As illustrated in FIG. 5, the hole 512 is sized and configured to accept the tip 522 and the spike 524 of an adjacent fastener 500. That is, during formation of the fasteners 500, as discussed in further detail below, the tip 522 and the spike 524 of one fastener 500 enter the hole 512 of an adjacent fastener 500, and sandwich tissue “T” (e.g., two layers of tissue) between the base 510 and a middle portion 526 of the leg 520 (FIG. 5). Further, the fasteners 500 are oriented such that they form a ring through tissue as each formed fastener 500 is engaged with two adjacent formed fasteners 500, as shown in FIG. 5. Additionally, in aspects, the spike 524 has sufficient flexibility to compress as it enters the hole 512 in the base 510, and has sufficient rigidity to hinder or prevent removal of the spike 524 from the hole 512.

With reference to FIGS. 6-9, and 11-13, details of the firing mechanism 300 are shown. The firing mechanism 300 is disposed within the end effector 200, and includes a ball ring 310 and a wedge ring 320. The ball ring 310 includes a plurality of balls 312 which form a ring within a proximal portion of the end effector 200. Each ball 312 of the plurality of balls 312 is joined or otherwise engaged with two adjacent balls 312. In aspects, at least one elastic strip 330 is used to join adjacent balls 312 of the ball ring 310 such that the balls 312 are movable relative to each other (e.g., in a radially outward direction), and are biased into contact with each adjacent ball 312 (e.g., in a radially inward direction). FIG. 11 schematically shows adjacent balls 312 spaced apart from each other, and FIGS. 12 and 13 schematically show adjacent balls 312 in contact with each other.

The wedge ring 320 includes a plurality of wedges 322 which form a ring within the end effector 200, and distally of the ball ring 310. Each wedge 322 is generally triangular-shaped including any combination of arcuate and linear sides. Additionally, each wedge 322 is affixed to two adjacent wedges 322 such that the wedges 322 are fixed from movement relative to each other. The wedge ring 320 is rotatable about the axis “A-A” relative to the ball ring 310 between an initial, pre-fire position where a tip 324 of each wedge 322 is between adjacent balls 312 (e.g., FIG. 11), and a second position where the tip 324 of at least one wedge 322 contacts a distal portion 314 of one ball 312 (e.g., FIG. 12). When the wedges 322 are not between adjacent balls 312, the ball ring 310 contracts to the orientation where each ball 312 contacts two adjacent balls 312 (FIG. 12). The third actuator 26 (FIG. 1) is used to cause the rotation of the wedge ring 320, to help form the fasteners 500, and to help decrease a diameter of the end effector 200, as discussed in further detail below.

Additionally, in aspects, the wedge ring 320 is movable or translatable along the axis “A-A” relative to the ball ring 310 between a first position where the tip 324 of at least one wedge 322 contacts a distal portion 314 of one ball 312 (e.g., FIG. 12), and a second position where the tips 324 of the wedges 322 re disposed proximally of and out of contact with the balls 312 (e.g., FIGS. 9 and 13). In aspects, a fourth actuator may allow a user to control the translation of the wedge ring 320 relative to the ball ring 310.

The disclosure also includes methods of performing an end-to-end anastomosis through a single bodily opening using the surgical fastening device 10. Initially, and with reference to FIGS. 1 and 2, after a tissue lumen has been cut (e.g., to remove a portion of the lumen), the end effector 200 of the surgical fastening device 10 is inserted through an opening (e.g., trans-anally) of a patient. The elongated portion 40 enters the opening and its articulating pivot 42 allows or facilitates the elongated portion 40 to follow the curves of the tissue lumen until the end effector 200 reaches the distal portion of the tissue lumen. With particular reference to FIG. 2, once the end effector 200 is in or near its desired, approximated position, the first actuator 22 is used to rotate the screw drive 250 in a first direction such that the end effector 200 moves away from the cartridge assembly 110. The desired distance between the end effector 200 and the cartridge assembly 110 can be confirmed by viewing the indicia 252a and/or 252b using a laparoscopic camera, for instance.

Once the appropriate position of the end effector 200 is confirmed, the second actuator 24 is used to apply an appropriate amount of suction to the tissue “T” via the proximal vacuum slots 112 of the cartridge assembly, the distal vacuum slots 212 of the end effector 200, the ring-like space between the screw drive 250 and the inner wall of the cartridge assembly 110, and/or the ring-like space between the screw drive 250 and the inner wall of the end effector 200.

As shown in FIGS. 2 and 3, the suction pulls the tissue “T” into contact with the end effector 200 and the cartridge assembly 110. Additionally, the suction pulls the tissue “T” into the area between the end effector 200 and the cartridge assembly 110, which may be helpful for formation of anastomotic donuts. Additionally or alternatively, the distal tissue and/or the proximal tissue may be positioned intracorporeally using surgical graspers, for instance. Next, while the suction is being applied to the tissue “T,” the first actuator 22 is used to rotate the screw drive 250 in a second direction such that the end effector 200 moves toward the cartridge assembly 110, thereby compressing two layers of tissue “T” between the end effector 200 and the cartridge assembly 110, as shown in FIG. 3. After the two layers of tissue “T” are compressed between the end effector 200 and the cartridge assembly 110, the second actuator 24 can be used to reduce or remove the suction, if desired.

The movement of the end effector 200 to the approximated position (FIG. 3), also moves the balls 312 of the firing mechanism 300 into contact with the fasteners 500 within the cartridge assembly 110 (FIG. 6). After initial contact between the balls 312 and the fasteners 500, continued proximal movement of the end effector 200 relative to the cartridge assembly 200 causes at least a partial formation of the fasteners 500. That is, each ball 312 exerts a proximally-directed force against a portion of the leg 520 of one fastener 500. This force causes at least the tip 522 of the leg 520 to enter the hole 512 of an adjacent fastener 500 (FIG. 5).

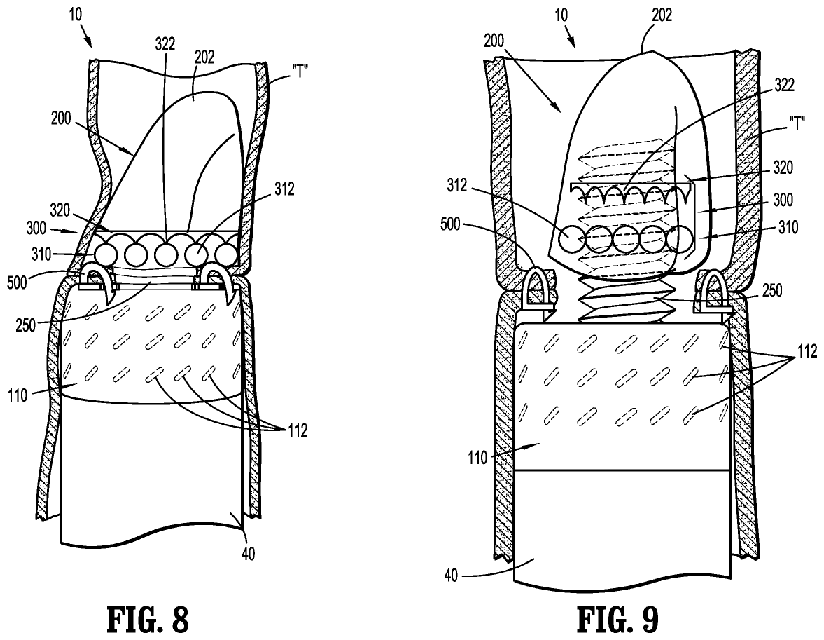

Next, to help ensure that the spike 524 of each fastener 500 also enters the hole 512 of an adjacent fastener 500 (i.e., to completely form the fasteners 500), the third actuator 26 is used to rotate the wedge ring 320 relative to the ball ring 310. This rotation causes the tip 324 of each wedge 322 to rotate away from its position between two adjacent balls 312. The rotation also causes the tip 324 and a side wall 325 of the wedges 322 to push the balls 312 further proximally until the tip 324 of at least one wedge contacts the distal end 314 of one ball 312. As the balls 312 are pushed farther proximally, the tips 522 of each fastener 500 are correspondingly pushed proximally, thereby ensuring the spike 524 of each fastener 500 is within the hole 512 of an adjacent fastener 500 (FIGS. 5 and 8). To help ensure each fastener 500 is properly formed, the user can continue to rotate (in one direction or in two directions) the wedge ring 320 relative to the ball ring 310 using the third actuator 26.

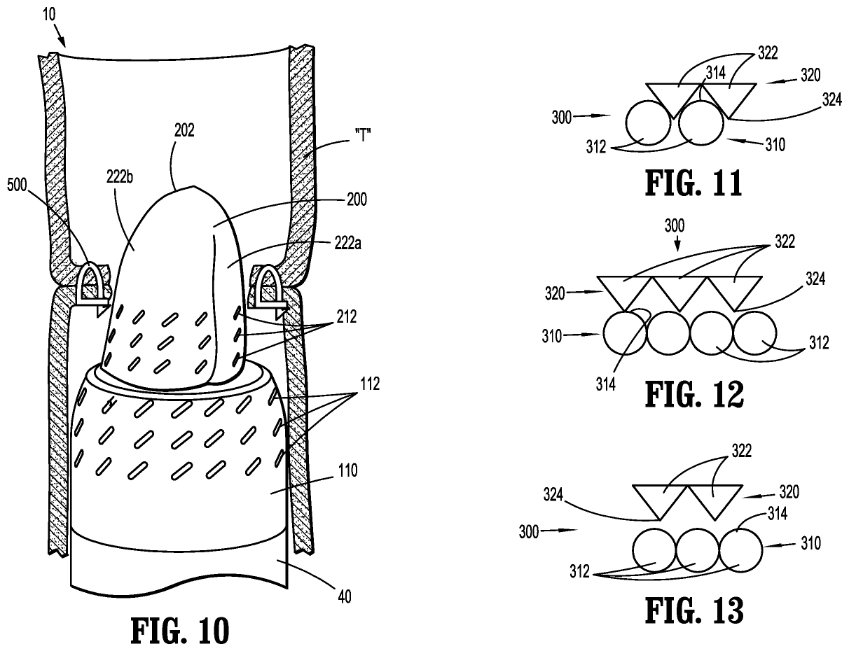

Additionally, as the wedge ring 320 is rotated relative to the ball ring 310, the wedges 322 are no longer between adjacent balls 312, thereby allowing the ball ring 310 to compress via the elastic strip 330 which joins adjacent balls 312, for instance. This compression of the ball ring 310 also allows the end effector 200 to compress or reduce the size of its outer dimension (e.g., perimeter or circumference). That is, the end effector includes a pair of wings 222a, 222b (FIG. 10) or folded sections. The wings 222a, 222b are biased to the folded or compressed orientation (FIG. 10), which results in the end effector 200 having a reduced outer dimension. When the ball ring 310 is in its initially, expanded orientation, the wings 222a, 222b of the end effector 200 are in the expanded or unfolded orientation.

Following the formation of the fasteners 500, and while the wings 222a, 222b of the end effector 200 are in their folded orientation, the surgical fastening instrument 10 can be removed from the bodily opening without unduly stressing the fastened tissue “T.”

While the above description contains many specifics, these specifics should not be construed as limitations on the scope of the present disclosure, but merely as illustrations of various aspects thereof. Therefore, the above description should not be construed as limiting, but merely as exemplifications of various aspects. Those skilled in the art will envision other modifications within the scope and spirit of the claims appended hereto.

What is claimed is:

1. A surgical fastening device, comprising:
a handle assembly;
an elongated portion extending distally from the handle assembly;
a shell assembly disposed adjacent a distal portion of the elongated portion, the shell assembly including a cartridge assembly and an end effector;
a screw drive defining a longitudinal axis, a distal portion of the screw drive engaged with the end effector, wherein rotation of the screw drive about the longitudinal axis relative to the elongated portion causes longitudinal translation of the end effector relative to the cartridge assembly; and
a firing mechanism disposed at least partially within the end effector, wherein a predetermined amount of proximal movement of the end effector relative to the cartridge assembly is configured to at least partially form fasteners disposed in the cartridge assembly.

2. The surgical fastening device according to claim 1, wherein the firing mechanism includes a ball ring having a plurality of balls, and a wedge ring having a plurality of wedges.

3. The surgical fastening device according to claim 2, wherein a tip of each wedge of the plurality of wedges of the wedge ring is positionable between adjacent balls of the plurality of balls of the ball ring.

4. The surgical fastening device according to claim 3, wherein the wedge ring is rotatable about the longitudinal axis relative to the ball ring.

5. The surgical fastening device according to claim 4, wherein rotation of the wedge ring relative to the ball ring causes a tip of at least one wedge of the plurality of wedges to move from a first position between adjacent balls of the plurality of balls, to a second position in contact with a distal end of one ball of the plurality of balls.

6. The surgical fastening device according to claim 2, wherein each ball of the plurality of balls is movable from a first position where each ball of the plurality of balls is free from contact with adjacent balls of the plurality of balls, to a second position where each ball of the plurality of balls contacts two adjacent balls of the plurality of balls.

7. The surgical fastening device according to claim 6, wherein each ball of the plurality of balls is biased toward the second position.

8. The surgical fastening device according to claim 6, wherein each wedge of the plurality of wedges is fixed from movement relative to adjacent wedges of the plurality of wedges.

9. The surgical fastening device according to claim 1, further comprising a tube extending at least partially through the elongated portion, wherein an external surface of the end effector includes a plurality of distal vacuum slots disposed in fluid engagement with the tube, and wherein an external surface of the cartridge assembly including a plurality of proximal vacuum slots disposed in fluid engagement with the tube.

10. The surgical fastening device according to claim 1, wherein the cartridge assembly includes a plurality of fasteners therein, wherein each fastener of the plurality of fasteners is configured to be secured to one adjacent fastener of the plurality of fasteners.

11. The surgical fastening device according to claim 10, wherein each fastener of the plurality of fasteners includes a base having a curved leg extending therefrom, the curved leg including a tip and a spike, and wherein the tip and the spike of the curved leg are configured to enter a hole of an adjacent fastener of the plurality of fasteners during formation of the plurality of fasteners.

12. A shell assembly of a surgical fastening device, comprising:
a cartridge assembly configured to house a plurality of fasteners, and including a plurality of proximal vacuum slots;
an end effector configured to move toward and away from the cartridge assembly, and including a plurality of distal vacuum slots, the proximal vacuum slots being in fluid communication with the distal vacuum slots; and
a firing mechanism disposed at least partially within the cartridge assembly, the firing mechanism including a ball ring having a plurality of balls, and a wedge ring having a plurality of wedges.

13. The shell assembly according to claim 12, wherein the wedge ring is rotatable relative to the ball ring between a first position where a tip of at least one wedge of a plurality of wedges of the wedge ring is between adjacent balls of a plurality of balls of the ball ring, and a second position where the tip of the at least one wedge is in contact with a distal end of one ball of the plurality of balls.

14. The shell assembly according to claim 12, wherein the ball ring includes a plurality of balls, each ball of the plurality of balls is movable from a first position where each ball of the plurality of balls is free from contact with adjacent balls of the plurality of balls, to a second position where each ball of the plurality of balls contacts two adjacent balls of the plurality of balls.

15. The shell assembly according to claim 14, wherein the wedge ring includes a plurality of wedges, each wedge of the plurality of wedges is fixed from movement relative to adjacent wedges of the plurality of wedges.

16. The shell assembly according to claim 12, wherein the cartridge assembly includes a plurality of fasteners therein, wherein each fastener of the plurality of fasteners is configured to be secured to one adjacent fastener of the plurality of fasteners.

17. A method of performing an end-to-end anastomosis through a single opening, comprising:
inserting a distal portion of a surgical fastening device through an opening to access target tissue;
positioning an end effector of the distal portion of the surgical fastening device at least partially within a distal tissue lumen;
positioning a cartridge assembly of the distal portion of the surgical fastening device at least partially within a proximal tissue lumen;
applying suction through the end effector such that portions of the distal tissue lumen are pulled into engagement with the end effector;
applying suction through the cartridge assembly such that portions of the proximal tissue lumen are pulled into engagement with the cartridge assembly;
moving the end effector towards the cartridge assembly;
compressing target tissue between the cartridge assembly and the end effector; and
moving a leg of a fastener disposed within the cartridge assembly through the target tissue and into engagement with an adjacent fastener within the cartridge assembly.

18. The method according to claim 17, further including rotating a ball ring within the end effector relative to a wedge ring within the end effector.

19. The method according to claim 18, wherein the ball ring including a plurality of balls, and further including biasing each ball of the plurality of balls radially inward.

20. The method according to claim 17, wherein the end effector defines an outer dimension, and further including reducing the outer dimension of the end effector, followed by removing the end effector from the single opening.
